(19) United States
(12) Patent Application Publication
(10) Pub. No.: US 2025/0198078 A1
(43) Pub. Date: Jun. 19, 2025

(54) Deodorizing Laundry Hamper

(71) Applicant: Brandon Blount, Los Angeles, CA (US)

(72) Inventor: Brandon Blount, Los Angeles, CA (US)

(21) Appl. No.: 18/538,285

(22) Filed: Dec. 13, 2023

(51) Int. Cl. D06F 95/00; A61L 2/22; A61L 2/26; B05B 9/04; B65D 25/02; B65D 43/16; B65D 53/02

(52) U.S. Cl. CPC D06F 95/002; A61L 2/22; A61L 2/26; B05B 9/0403; B65D 25/02; B65D 43/163; B65D 53/02; A61L 2202/121; A61L 2202/122; A61L 2202/15; A61L 2202/26; B65D 2543/00194; B65D 2543/00972

(57) ABSTRACT

A deodorizing laundry hamper includes a body and a lid pivotably attached thereto, each of which comprises an airtight material. A seal arrangement is positioned between the lid and the body to seal the deodorizing laundry hamper in an airtight manner. The hamper further includes a deodorizing arrangement that delivers deodorizing media into the interior of the body to deodorize the body and/or laundry articles therein.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to a laundry hamper and more particularly pertains to a new airtight laundry hamper comprising a deodorizing arrangement. Laundry hampers are used to temporarily store articles, such as used or dirty clothing, towels, sheets, or similar objects, after use but prior to cleaning or washing. Such articles can be contaminated with odor-causing materials that produce a malodor. The malodors are not only offensive to persons in the vicinity of the laundry hamper, but could also settle into and remain in the surrounding area or room, as well as into any other articles in the laundry hamper, especially if the articles are in the laundry hamper for extended periods, such as days or even weeks.

### (2) Description of Related Art Including Information Disclosed Under 37 CFR 1.97 and 1.98

The prior art relates to airtight containers and receptacles, such as garbage cans, laundry hampers, and similar receptacles that trap odors within the container. However, none of the prior art discloses a laundry hamper having an airtight body, airtight lid, and a deodorizing arrangement.

### BRIEF SUMMARY OF THE INVENTION

An embodiment of the disclosure meets the needs presented above by generally comprising a laundry hamper having an airtight body, airtight lid, and a deodorizing arrangement. In accordance with at least one possible embodiment, a laundry hamper has an airtight body and an airtight lid. Neither the lid nor the body of the laundry hamper has any gaps or openings to permit airflow therethrough. In addition, the lid and/or the rim of the body includes a sealing structure to produce an airtight seal between the lid and the body when the lid is closed. The laundry hamper therefore can trap malodors therein to prevent users from having to smell the malodors or the surrounding area from being contaminated by malodors.

In accordance with at least one possible embodiment, the laundry hamper further includes a deodorizing arrangement. The deodorizing arrangement can be designed to either actively or passively release deodorizing media, such as fragrances and/or fresheners and/or sterilization media to kill odor-causing bacteria and other biological agents, into the body of the laundry hamper. In accordance with at least one possible embodiment, the deodorizing arrangement can be automatic and controlled by a control arrangement, such as a microprocessor. Such a control arrangement could operate the deodorizing arrangement on a schedule and/or when certain conditions are met, such as the lid being closed.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a front perspective view of a deodorizing laundry hamper according to an embodiment of the disclosure.

FIG. 2 is a front perspective view of the deodorizing laundry hamper with the lid open.

FIG. 3 is a rear perspective view of the deodorizing laundry hamper.

FIG. 4 is a top view of the deodorizing laundry hamper.

FIG. 5 is a cross-sectional view of a portion of the deodorizing laundry hamper.

FIG. 6 is a front view of the deodorizing laundry hamper with articles therein.

FIG. 7 is a front perspective view of a deodorizing laundry hamper with the lid open according to at least one other embodiment of the disclosure.

### DETAILED DESCRIPTION OF THE INVENTION

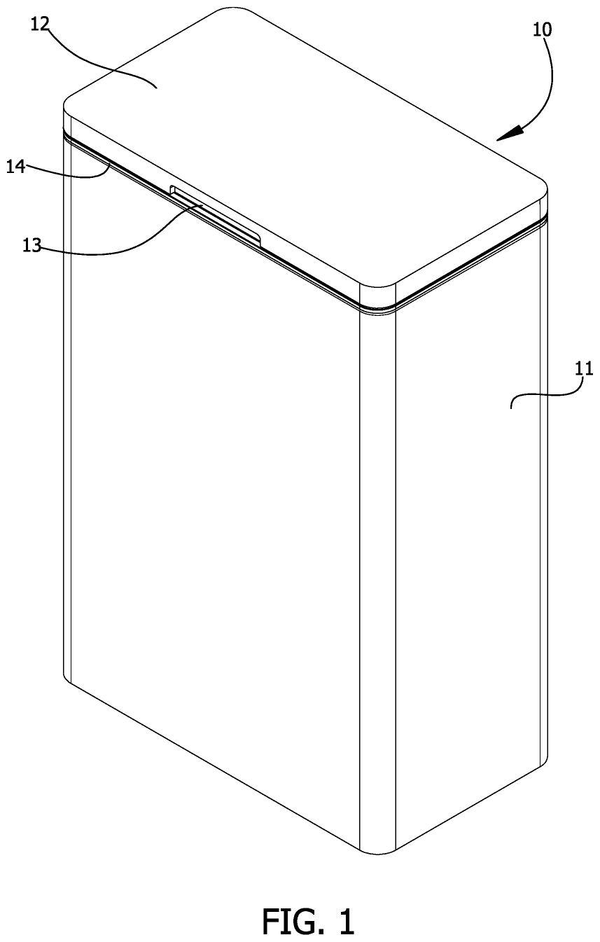

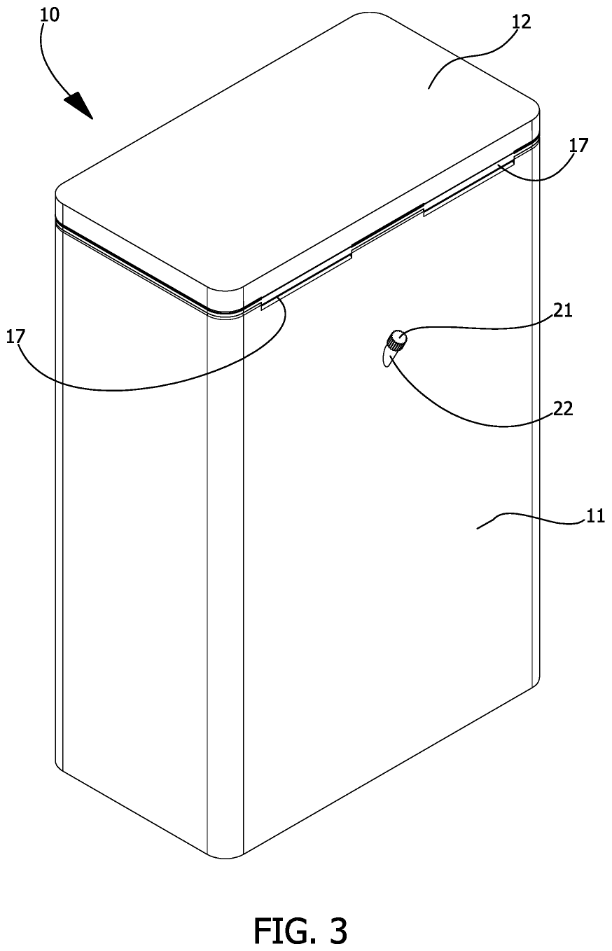

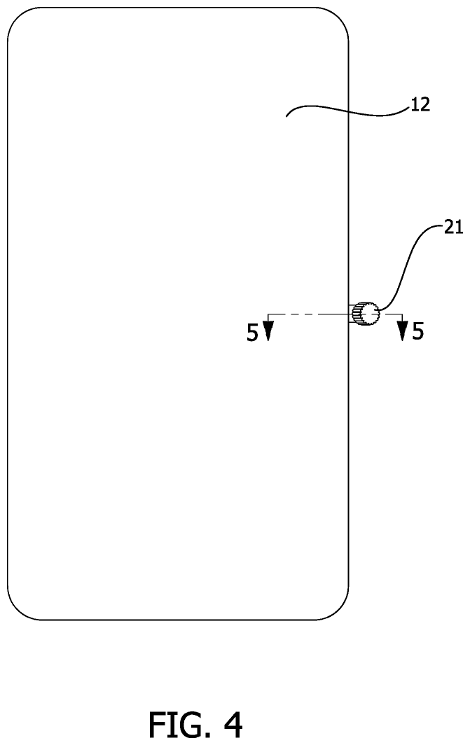

With reference now to the drawings, and in particular to FIGS. 1 through 7 thereof, a new laundry hamper embodying the principles and concepts of an embodiment of the disclosure and generally designated by the reference numeral 10 will be described.

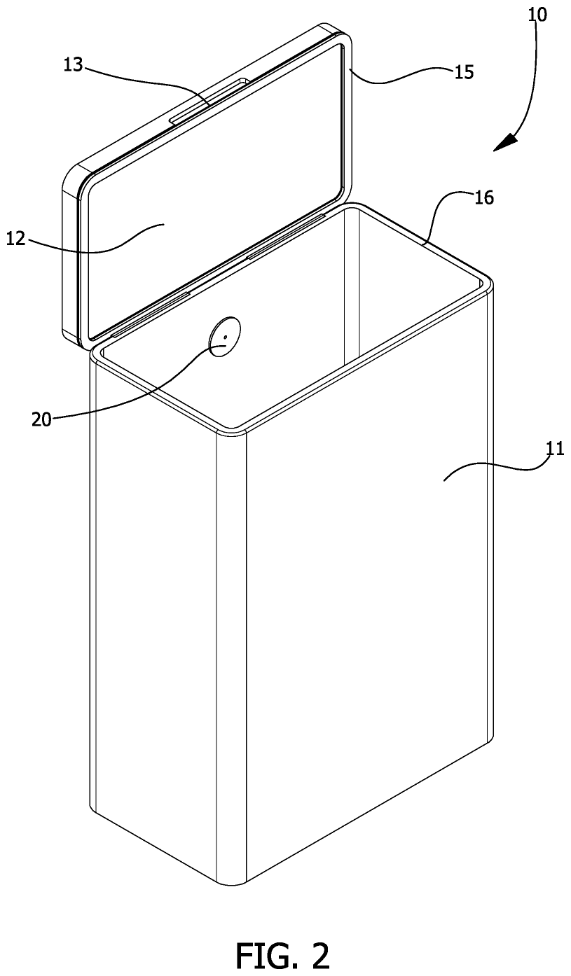

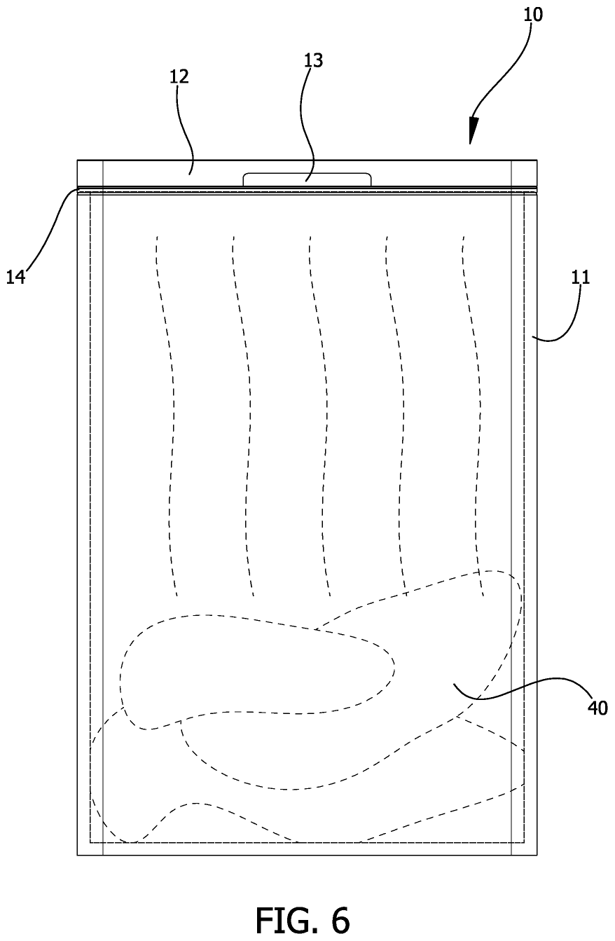

As best illustrated in FIGS. 1 through 7, the deodorizing laundry hamper 10 generally comprises a body 11 and a lid 12 pivotably connected thereto by hinges 17. Each of the body 11 and lid 12 are made of airtight material. The lid 12 includes a handle 13. A seal arrangement 14 is located between the body 11 and the lid 12 to form an airtight seal. As seen in FIG. 2, the lid 12 can be pivoted upwardly to provide access to the interior of the body 12 for inserting and removing laundry articles 40 (see FIG. 6), such as used and/or soiled clothing, towels, and linens. The seal arrangement 14 can be made of two parts: a lid portion 15 located at the perimeter edge of the lid 12 and a body portion 16 located at the perimeter edge of the opening in the body 11.

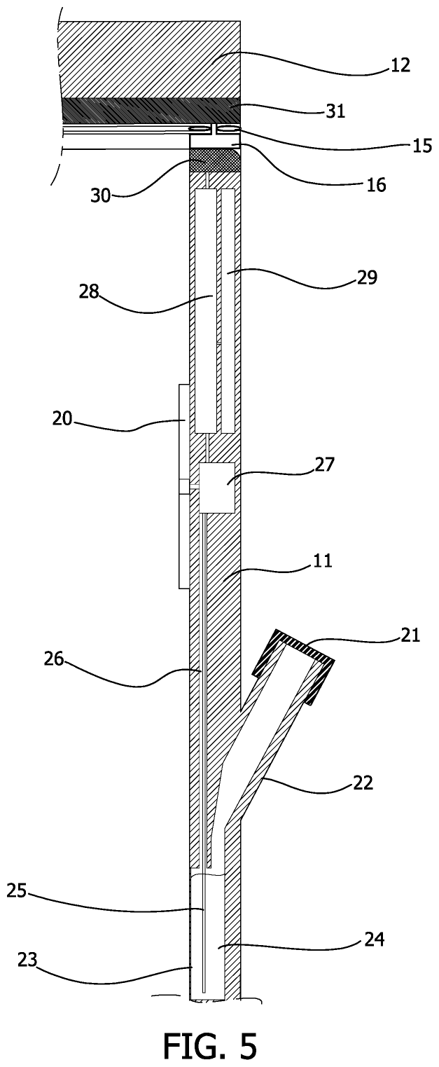

Located within the body 11 is a dispenser 20 for dispensing or spraying deodorizing media into the interior of the deodorizing laundry hamper 10 to deodorize the contents thereof. On the back of the body 11, a cap 21 covers a filling spout 22. As seen in FIG. 5, the filling spout 22 is connected to a reservoir 23 or chamber designed to contain deodorizing media 24, such as a sprayable liquid. A tube 25 projects into the deodorizing media 24. The tube 25 is located in a channel 26 within the side of the body 11. A pump 27 is connected to the tube 25 to draw the deodorizing media 24 out of the reservoir 23. The pump 27 is operatively connected to a controller 28, such as a computer controller or microprocessor, which controls the operation of the pump 27. The controller 28 is powered by a power source 29, such as a battery. In operation, the controller 28 activates the pump 27, either on demand or according to a schedule, to dispense deodorizing media out of the dispenser 20 into the interior of the body 11. In the embodiment shown in FIG. 5, the controller 28 is operatively connected to a magnetic switch 30, which is located in the wall of the body 11 and controls the actuation of the controller 28. A magnet 31 is located in the lid 12, which magnet 31 can actuate the magnetic switch 30. In this embodiment, when the lid 12 is open or raised, the magnet 31 is located a distance from the magnetic switch 30, and thus the magnet 31 cannot actuate the magnetic switch 30 and thus the controller 28. When the lid 12 is closed, the magnet 31 is close enough to the magnetic switch 30 to actuate it and thereby actuate the controller 28. In effect, deodorizing media 24 can only be dispensed when the lid 12 is closed and not when the lid 12 is open.

Also seen in FIG. 5 is a possible embodiment of the seal arrangement 14. Specifically, the lid portion 15 is in the form of a sealing body split into two parts. The body portion 16 is in the form of a sealing body that includes a projection designed to project into the space in the lid portion 15. The lid portion 15 and the body portion 16 engage with one another to form the airtight seal arrangement 14. It should be understood that the embodiment of the seal arrangement 14 shown in FIG. 5 is exemplary of one possible embodiment, and thus any suitable sealing arrangement that produces an airtight seal could be substituted and is disclosed by this application.

In use, a user fills the deodorizing laundry hamper 10 with laundry articles 40 (see FIG. 6), such as clothing, linens, and towels. The user then closes the lid 12 to thereby seal the laundry articles 40 inside the hamper 10 in an airtight manner. The dispenser 20, under the control of the controller 28, then dispenses deodorizing media 24, either on demand or according to an automatic schedule. The deodorizing media 24 deodorizes the interior of the hamper 10 and/or the laundry articles 40.

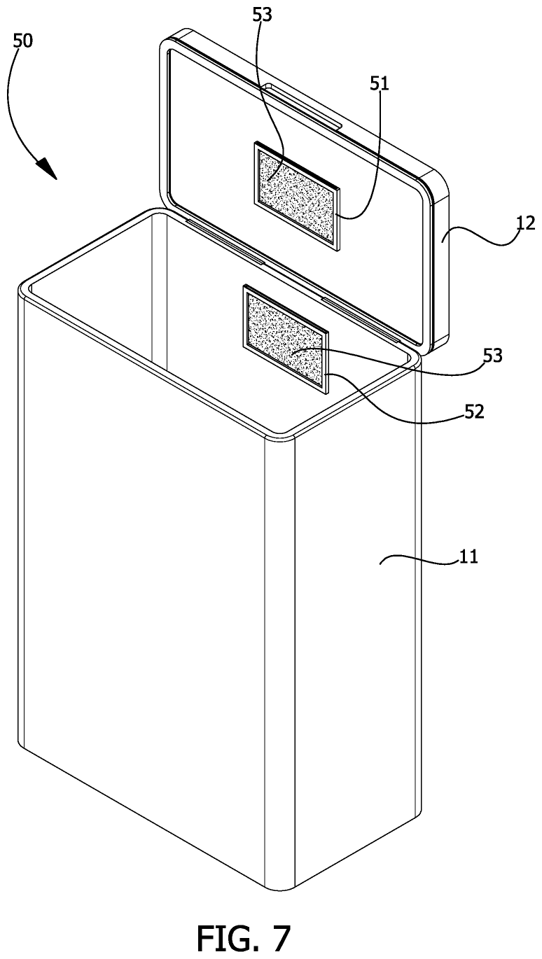

As seen in FIG. 7, the system for actively dispensing deodorizing media 24 is replaced by a passive system. In the embodiment shown, a deodorizing laundry hamper 50 includes a first holder 51 on the lid 12 and a second holder 52 on the body 11. These holders 51, 52 are designed to hold passive deodorizing structures 53, such as sheets or cartridges containing a deodorizing media. In operation, the deodorizing structures 53 continuously release deodorizing media into the interior of the body 11. When the deodorizing structures 53 are used up or low on deodorizing media, they can be replaced with new or fresh deodorizing structures 53.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A deodorizing laundry hamper comprising:
a body being configured to temporarily contain laundry articles therein;
a lid being pivotably attached to said body and configured to open and close an opening of said body;
each of said body and said lid comprising airtight material;
a seal arrangement being disposed at the edge of said lid and the edge of said body to seal said deodorizing laundry hamper in an airtight manner; and
a deodorizing arrangement being configured to deliver deodorizing media into the interior of said body to deodorize at least one of the interior of said body and laundry articles therein.

2. The deodorizing laundry hamper according to claim 1, wherein said deodorizing arrangement comprises a dispensing arrangement configured to actively dispense deodorizing media into the interior of said body, either on demand or according to an automated schedule.

3. The deodorizing laundry hamper according to claim 2, wherein said dispensing arrangement comprises:
a dispensing aperture mounted to a wall of said body facing the interior of said body to dispense or spray deodorizing media into the interior of said body;
a pump operatively connected to said dispensing aperture;
a reservoir configured to contain a supply of deodorizing media and operatively connected to said pump; and
a controller operatively connected to said pump and configured to actuate said pump to pump deodorizing media from said reservoir and out through said dispensing aperture.

4. The deodorizing laundry hamper according to claim 3, wherein each of said pump, reservoir, and said controller are disposed within a wall of said body.

5. The deodorizing laundry hamper according to claim 4, further comprising an actuation arrangement configured to permit actuation of said dispensing arrangement upon said lid being closed and prevent actuation of said dispensing arrangement upon said lid being open.

6. The deodorizing laundry hamper according to claim 5, wherein said actuation arrangement comprises:
a magnetic switch mounted in or on a portion of said body adjacent said lid;
said magnetic switch is operatively connected to said controller to actuate said controller; and
a magnet mounted on or in said lid and configured to actuate said magnetic switch upon said lid being closed and said magnet being sufficiently close to said magnetic switch to actuate said magnetic switch.

7. The deodorizing laundry hamper according to claim 6, wherein:
said body comprises a filling spout disposed to project out of a wall of said body;
said filling spout is connected to said reservoir; and
said filling spout comprises a cap configured to be removed to open said filling spout and permit the filling of said reservoir with deodorizing media.

8. The deodorizing laundry hamper according to claim 7, wherein said seal arrangement comprises:
a lid portion disposed at the perimeter edge of said lid;
a body portion disposed at the perimeter edge of said opening in said body; and
said lid portion and said body portion are configured to engage with one another upon said lid being closed to create an airtight seal.

9. The deodorizing laundry hamper according to claim 2, further comprising an actuation arrangement configured to permit actuation of said dispensing arrangement upon said lid being closed and prevent actuation of said dispensing arrangement upon said lid being open.

10. The deodorizing laundry hamper according to claim 9, wherein said actuation arrangement comprises:
a magnetic switch mounted in or on a portion of said body adjacent said lid;
said magnetic switch is operatively connected to said controller to actuate said controller; and
a magnet mounted on or in said lid and configured to actuate said magnetic switch upon said lid being closed and said magnet being sufficiently close to said magnetic switch to actuate said magnetic switch.

11. The deodorizing laundry hamper according to claim 3, wherein:
said body comprises a filling spout disposed to project out of a wall of said body;
said filling spout is connected to said reservoir; and
said filling spout comprises a cap configured to be removed to open said filling spout and permit the filling of said reservoir with deodorizing media.

12. The deodorizing laundry hamper according to claim 1, wherein said seal arrangement comprises:
a lid portion disposed at the perimeter edge of said lid;
a body portion disposed at the perimeter edge of said opening in said body; and
said lid portion and said body portion are configured to engage with one another upon said lid being closed to create an airtight seal.

13. The deodorizing laundry hamper according to claim 1, wherein said deodorizing arrangement comprises at least one deodorizing structure configured to continuously and passively dispense deodorizing media into the interior of said body.

14. The deodorizing laundry hamper according to claim 13, wherein:
said deodorizing arrangement comprises at least one of:
a first holder disposed on an interior surface of said lid; and
a second holder disposed on an interior surface of said body;
each of said first holder and said second holder is configured to hold one of said at least one deodorizing structure.

15. The deodorizing laundry hamper according to claim 14, wherein said seal arrangement comprises:
a lid portion disposed at the perimeter edge of said lid;
a body portion disposed at the perimeter edge of said opening in said body; and
said lid portion and said body portion are configured to engage with one another upon said lid being closed to create an airtight seal.

16. A method of deodorizing in a deodorizing laundry hamper, said method comprising the steps of:
opening a lid made of airtight material and being pivotably attached to a body of the laundry hamper made of airtight material;
placing laundry articles into said body;
closing said lid and forming a seal at the edge of said lid and the edge of said body to seal said deodorizing laundry hamper with a seal arrangement in an airtight manner; and
operating a deodorizing arrangement to deliver deodorizing media into the interior of said body and deodorizing at least one of the interior of said body and laundry articles therein.

17. The method according to claim 16, wherein said step of operating said deodorizing arrangement comprises actuating a dispensing arrangement configured to actively dispense deodorizing media into the interior of said body, either on demand or according to an automated schedule.

18. The method according to claim 17, wherein said step of actuating said dispensing arrangement comprises:
activating a controller disposed in said body and operatively connected to a pump disposed in said body;
pumping deodorizing media from a reservoir disposed in said body and to a dispensing aperture mounted to a wall of said body facing the interior of said body; and
dispensing deodorizing media out of said dispensing aperture and into the interior of said body.

19. The method according to claim 18, wherein said method further comprises:
closing said lid to permit actuation of said dispensing arrangement; and
opening said lid being to prevent actuation of said dispensing arrangement.

20. The method according to claim 19, wherein said step of actuating said dispensing arrangement comprises:
closing said lid and thereby moving a magnet mounted on or in said lid sufficiently close to a magnetic switch mounted in or on a portion of said body adjacent said lid and operatively connected to said controller; and
actuating said magnetic switch and thereby actuating said controller.
